(19) United States
(12) Patent Application Publication
(10) Pub. No.: US 2012/0203130 A1
(43) Pub. Date: Aug. 9, 2012

(54) Tinnitus Therapy Device

(76) Inventor: Armin Bernhard, Wien (AT)

(21) Appl. No.: 13/386,298

(22) PCT Filed: Jul. 15, 2010
(86) PCT No.: PCT/EP2010/060256

§ 371 Date: Jan. 20, 2012

(30) Foreign Application Priority Data
Jul. 22, 2009 (DE) 10 2009 034 491.8

(51) Int. Cl. A61N 2/02; A61B 5/0476

(52) U.S. Cl. 600/544

(57) ABSTRACT

The invention relates to a device for tinnitus therapy having an EEG device, particularly a Q-EEG device and a head piece having at least one applicator generating a low-frequency electromagnetic field in the frequency range 1-100 Hz and having a field strength below 20 mT.

### CROSS-REFERENCE TO RELATED APPLICATIONS

This application is the U.S. National Phase of International Application No. PCT/EP2010/060256, entitled “Tinnitus Therapy Device,” filed Jul. 15, 2010, which, in turn, claims the benefit of German Patent Application No. 10 2009 034 491.3, filed Jul. 22, 2009, the disclosures of which are hereby incorporated by reference in their entireties.

### TECHNICAL FIELD OF THE INVENTION

The present invention generally relates to a tinnitus therapy device intended to reduce and/or suppress tinnitus.

### BACKGROUND OF THE INVENTION

The tinnitus research thus far approached this symptom through monocausal strategies; but till today the desired successes is still lacking. Based on the present knowledge a multi-functional and interdisciplinary approach is being considered as the basis for our research and product development.

The tinnitus issue is considered to be very complex, since tinnitus is perceived differently (tinnitus duration, and differing tinnitus noise) and each individual is affected differently by the ringing of the ear. Thus far medication and infusion therapy were employed for treating tinnitus. These strategies showed positive effects only in patients suffering from an acute tinnitus. Additionally psychological procedures (e.g. behavior therapy, explanation of the facts, counseling), acoustic masking (e.g. with a noise generator in the form of a hearing aid), different methods of relaxation and alternative procedures e.g. electrical stimulation have been employed for the treatment of tinnitus.

### SUMMARY OF THE INVENTION

The invention relates to the development of a technically optimized diagnostical system (Cermag) and a therapy device (Certis) with the intent to reduce and/or suppress tinnitus, all based on the concept/approach of the transcranial electromagnetic stimulation. Cermag is based on a quantitative EEG (Q-EEG), employing only very specific electrode sites. EEG stands for electroencephalography. Q-EEG dare submitted to a FFT analysis (Fourier), thus the EEG signals are converted into power spectrum. The analysis of the Q-EEG data, being recorded under the influence of magnetic field—varying over a certain frequency range—, allows a specific and optimized therapy through an applicator being placed at certain brain sites and/or spinal cord. The applied magnetic field leads to measurable positive effects in these patients. The transcranial magnetic stimulation of low field strength leads via frequency and direction alterations to changes of the Q-EEG-signature.

### BRIEF DESCRIPTION OF THE DRAWINGS

FIG. 1 is a side view and top view of the placement of electrodes on the scalp of a patient in a standard 10-20 international pattern;

FIG. 2 is a second side view of the placement of electrodes on the scalp of a patient in the standard 10-20 international pattern;

FIG. 3 is an 8 second qualitative EEG showing the effectiveness of the therapy in a patient case study;

FIG. 4 is a map of the brain waves computed from the EEG data of FIG. 3;

FIG. 5 is a map of the power spectrum computed from the EEG data of FIG. 3;

FIG. 6 illustrates exemplary changes in beta power resulting from pulsed magnetic field therapy;

FIG. 7 shows exemplary changes in delta, theta, alpha and beta values between a patient with tinnitus and a patient in tinnitus remission resulting from pulsed magnetic field therapy;

FIG. 8 illustrates exemplary changes in an EEG for different patients, resulting from pulsed magnetic field therapy;

FIG. 9 illustrates exemplary changes in an EEG for different patients, resulting pulsed magnetic field therapy at different frequencies;

FIG. 10 illustrates exemplary maps of patient brain waves and power spectrum computed from EEG data obtained before and after utilizing the tinnitus therapy device of the present invention;

FIG. 11 illustrates acquired and calculated patient data to be displayed on a screen;

FIG. 12 is a Certis head device of the present invention having a head phone frame and a therapy element;

FIG. 13 is an alternative embodiment of the Certis head device of the present invention having a head phone frame with a rail system with several positions for adjusting the therapy element;

FIG. 14 is an alternative embodiment of the Certis head device of the present invention having a transverse handle attached thereto via a rail system, with two therapy elements attached to the transverse handle;

FIG. 15 is an alternative embodiment of the Certis head device of the present invention having a head band with two therapy elements fastened to a loop of the head band;

FIG. 16 is an alternative embodiment of the Certis head device of the present invention having a head band which can be opened on the inside to hang in therapy elements at different positions; and

FIG. 17 is a therapy device of the present invention attached to an ear clip, which is placed on the outside of an ear.

### DETAILED DESCRIPTION OF EMBODIMENTS OF THE INVENTION

The therapy equipment Certis consists of a head device and a base. Via the head device certain parts of the head, in particular the interior ear, hearing nerve, auditory pathway nuclei as well as parts of the brain and/or the spinal column or parts of the spinal column are stimulated with a low-frequency electrical and/or electromagnetic field in the frequency range—determined by the Q-EEG—via applicators (iron core coils). The portable therapy device Certis programmed with the patient-typical individualized parameters (frequency, field strength etc.) provides the individualized stimulation parameters for the magnetic field. This therapy device is characterized by two features, namely providing on one hand the magnetic field stimulation signals and on the other hand containing a high quality auditory system for playing music relevant for tinnitus therapy This stimulation leads to above described positive phenomena, which can be confirmed by the Q-EEG data analysis. The characteristic of this device is that both stimulation procedures can be offered simultaneously. In the therapy equipment incorporated is additionally a control unit. With the help of this control unit the patient can use the device. Due to the product properties the equipment enables humans, who are suffering from tinnitus, to pursue her or his daily private and or professional activities.

The equipment Certis comprises a mobile protocol data memory chip and an own data memory chip for the individualized parameters. The head device—stimulator—comprises a foam cube, in which the head is placed during treatment. Stimulation coils (applicators) are integrated in the head device. In combination with the questionnaire according to Goebel and Hiller the success of the therapy can be objectively measured.

Novelty, advantage and methods: Quantitative EEG and Tinnitus The recording of the electroencephalographic activity is performed by placing electrodes on the scalp in a standard international (10/20) pattern. In order to be able to register the weak electrical voltage fluctuations of the brain within the range of 1-200 microvolts (μV) silver chloride electrodes were employed. For the improvement of the impedance and resistivity values between scalp and electrode the electrode cups are filled with a special conduct paste.

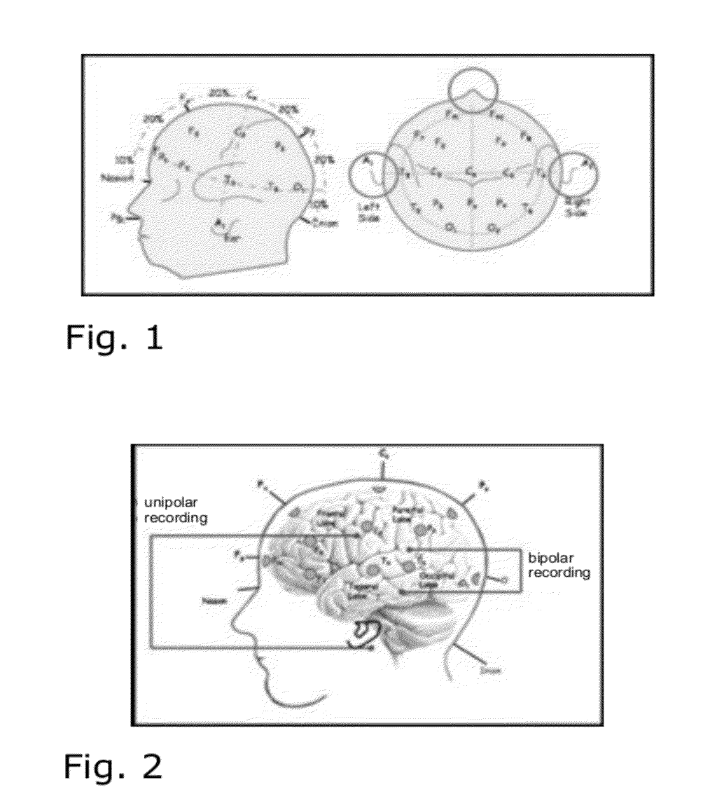

This standard system, the so-called 10-20-system, was developed by Jasper (1958) and is represented in the FIGS. 1 and 2.

In order to make the weak signals visible, very sensitive amplifiers are employed. Both, high and low-pass filters can suppress artifacts (noise) outside of the interesting EEG frequency ranges. The enhanced and filtered analog signals are converted into digital signal sequences. The sampling rate determines often the analog signal is quantified per second. The digital signal sequences are stored continuously on a recording system.

The recording of the EEG is performed in a sound proof and electrically shielded room. The EEG is recorded while the subject is sitting in a relaxing chair. The recording of the brain waves is performed at rest under the condition eyes open and eyes closed over a period of 10 minutes each. The recorded EEG data are visually inspected. All EEG epochs affected with artifacts such as muscle potentials, eye movements, electrode artifacts etc) are being deleted. Subsequently the artifact free EEG data (2 second epochs) are subjected to an FFT (fast Fourier transformation) analysis.

The EEG data are illustrated in form of brain maps, power spectrum and numerics. The statistical analysis was performed with the StatView Program.

With the help of special software the stored raw data can be presented in differing montages.

The EEG is divided into the following classical frequency bands (2-4 Hz (Delta), 4-7 Hz (Theta), 8-13 Hz (Alpha), 14-21 Hz Beta), 22-64 Hz Gamma), in 1 Hz bins, and in brain maps in order to illustrate the information hidden in the so-called background activity.

Tinnitus—measurable/visible in EEG?

The assessment of subjective Tinnitus is limited despite employing audiometrical and psychometrical procedures. We asked ourselves whether the Q-EEG contains typical related to the tinnitus and if these changes can be used as a base for tinnitus therapy and for the evaluation of the effectiveness of the therapy.

The Q-EEG data e.g. certain frequency bands are presented in form of brain maps (relative absolute power) or as spectral image (power or magnitude). We compared the Q-EEG data from 500 normal controls with 599 patients suffering from tinnitus. Tinnitus induced changes of the total average power. It could be shown, that male tinnitus sufferers exhibited a significantly reduced total average power when compared to normal male controls. For female tinnitus patients a significantly increased total average power was noted when compared to normal female controls. In the male group most changes were related to the alpha and theta bands and to a lesser degree to the delta band. In the female group alpha and beta bands were mostly affected.

The frequency parameter mapping revealed gender independently a delta and a theta focus in CZ extending to frontal regions. The power spectrum revealed that in temporal sites delta activity was increased compared to the alpha activity. Furthermore a tinnitus typical feature was identified. The observed temporal alpha suppression is often noticed throughout the sensory motor strip Tinnitus patients also exhibit an increased beta power in the temporo-central and left parietal region.

These data allowed us to establish a scoring system for tinnitus.

| Ranking | Item | Significance |
|---|---|---|
| 1 | Reduced total average power | 3 |
| 2 | Alpha suppression (T4) | 3 |
| 3 | Theta focus (CZ) | 3 |
| 4 | Beta-focus | 3 |
| | (temporo-centro-parietal) | |
| 5 | Alpha suppression (T3) | 3 |
| 6 | Delta focus (CZ) | 1 |
| 7 | T4Delta > T4 Alpha | 1 |
| 8 | T3Delta > T3 Alpha | 1 |

| Score | Tinnitus |
|---|---|
| 5 or 6 | perhaps |
| 7 to 11 | possible |
| 11 to 13 | very possible |
| from 14 up | certain |

The importance of the Tinnitus typical EEG signals was confirmed by the acute remission of tinnitus while recording the EEG. The positive effect of a Tinnitus masker with broadband noise shows up in the power spectrum as an increase of the alpha power, the negative effect as a leftward shift of the alpha frequency toward low frequency ranges.

Manual therapy of the atlanto-occipital and atlantoaxial joints which play a role in the pathogenetic mechanism of tinnitus, lead to a reduction of delta—theta activity and a rise of the alpha power.

In order to employ neurofeedback successfully the exact knowledge of the patient-typical tinnitus EEG features/signals must be known. Successful neurofeedback can be demonstrated either by quantitative EEG or with the help of the tinnitus questionnaire according to Goebel and Hiller, the SCL90R questionnaire and the BDI questionnaire.

The transcranial magnet stimulation of low field strength induces positive or negative changes of the EEG signature pending on its frequency and its direction. Its success can be demonstrated as we did for neurofeedback with the help of the quantitative EEG, the questionnaire according to Goebel and Hiller, the SCL90R questionnaire and the BDI questionnaire.

The practical significance of the QEEG data is that they permit to evaluate therapies, but at the same time they are the basis for the use of magnetic field therapy or neurofeedback. Furthermore, in tinnitus patients the EEG diagnostics permit to employ a reduced number and/or loci of electrodes.

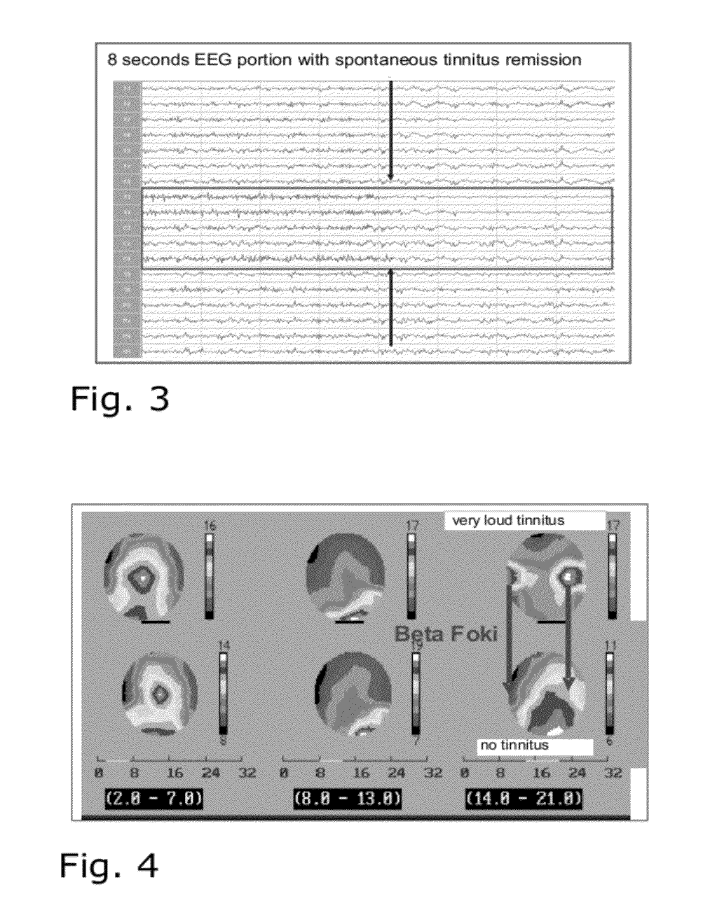

The quantitative computer-assisted EEG analysis shows, to which extent the electroencephalographic activities have changed. Q-EEG analysis technique allows to compute EEG data and to illustrate them in form of maps (EEG map o. spectral parameter map). The registration of the EEG is performed in accordance with the guidelines of the German society for clinical neurophysiology (19 positions according to the 10-20-system) and all these data are stored electronically. After visual inspection the FFT of the artifact-free EEG epochs (2 seconds each) was performed. The EEG data are presented in the form of brain maps, power spectra and numerics. The computation of the power values is performed for the frequency ranges: delta (0.5-4 Hz), theta (4-7 Hz), alpha (8-13 Hz) and beta (14-21 Hz) and gamma (22-64 Hz). In FIG. 3 on the basis a case report the effectiveness is illustrated:

Patient: Sex: male, age: 55 years, tinnitus duration: 38 months, Tinnitus on both sides.

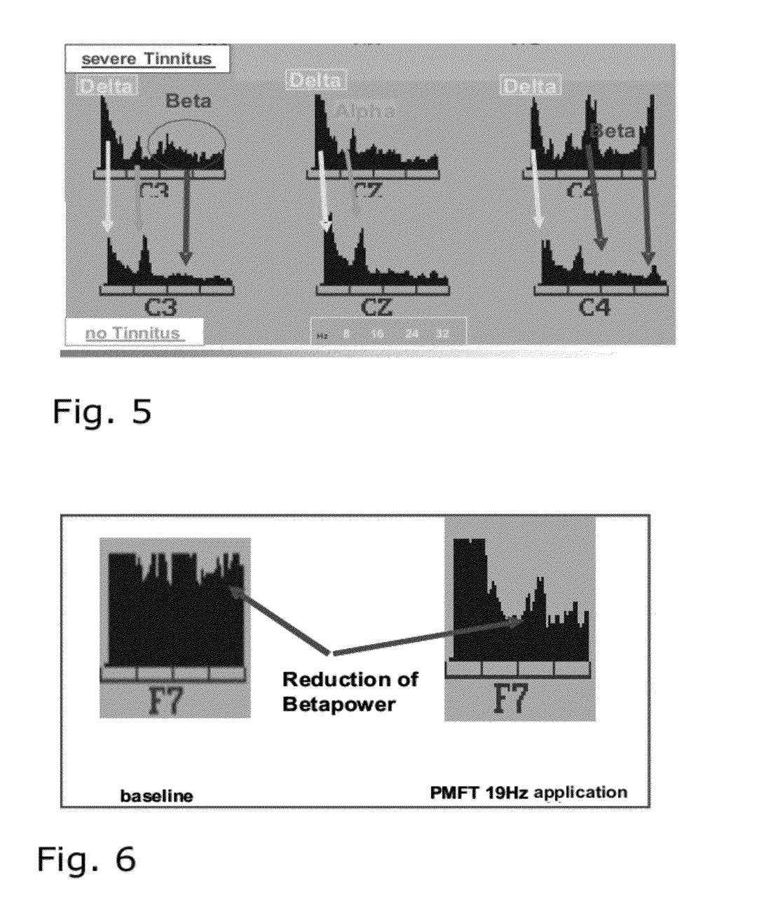

The appropriate brainmap is graphically represented in FIG. 4, the power spectrum in FIG. 5.

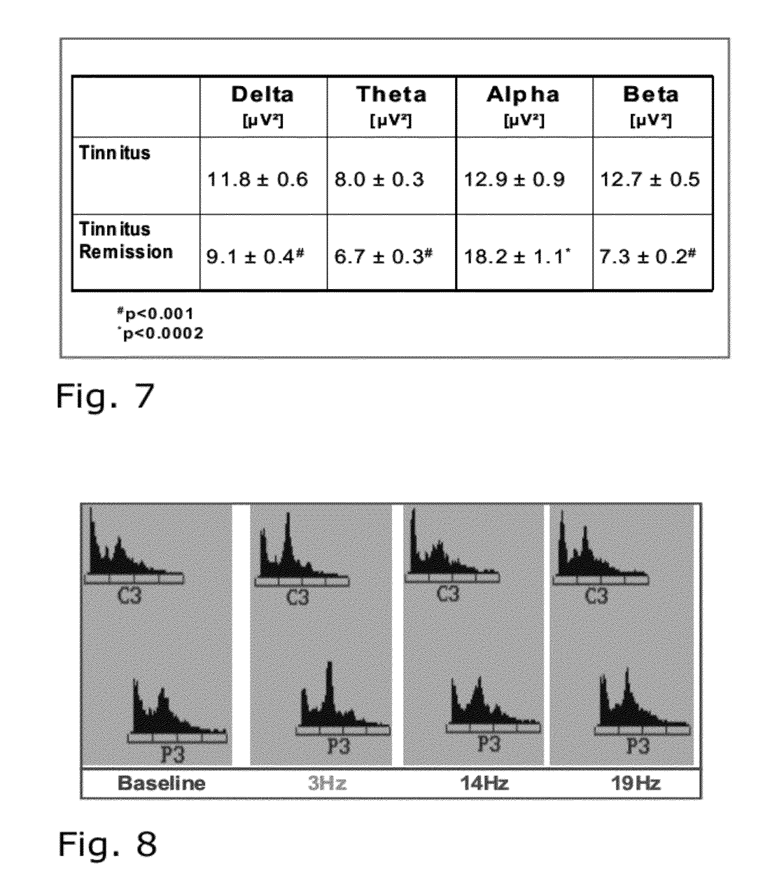

The computer—assisted evaluation of the EEG data furnished evidence that frequency-steered magnetic fields induce frequency dependent changes of the brain wave pattern in tinnitus patients. Changes of the beta power in respect to PMFT application (pulsed magnetic field therapy) is represented in FIGS. 6 and 7.

Summary:
1. Spontaneous remission of tinnitus correlates with changes of the EEG-profile.
2. significant increase in alpha power
3. significant reduction of delta, theta and beta power.
4. these data provide very important information for the magnetic field tinnitus therapy
5. tinnitus is demonstrable in Q-EEG
6. therapies can be evaluated for their effectiveness
7. only an individually designed therapy has a chance to be successful

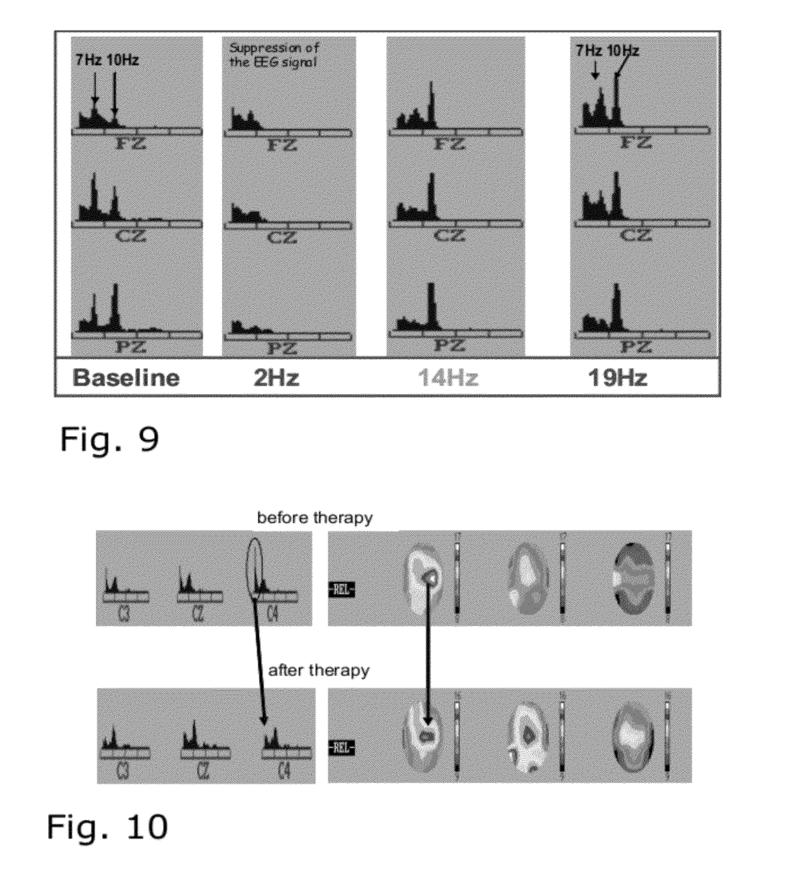

In FIGS. 8 and 9 different effects (positive/negative) on the EEG of different persons are illustrated.

Clinical and physiological data show that in patients suffering from a chronic tinnitus exhibit a focally increased brain activity. After applying a pulsed magnetic field therapy a scientific study revealed a significant increase in delta, theta and alpha power. These changes were mainly demonstrable in the frontal brain. The data point out that the application of the pulsed magnetic field therapy causes changes in the EEG signature, which again correlate with an alleviation of the tinnitus symptoms In the last years evidence could be furnished that tinnitus is a central event On the basis of the Q-EEG analysis it could be shown that tinnitus sufferers compared with healthy controls exhibit significant differences for delta/theta, alpha and beta waves. Computer-assisted EEG analysis demonstrated a significant reduction of the alpha power. Delta, theta and beta waves however were characterized by a significant increase of their power values. The present results point to the fact that the four frequency ranges are of significance for the pathogenetic mechanism of tinnitus. Based on this knowledge new tinnitus therapies can be developed.

Summary:

With the help of the Q-EEG subjective feelings of a patient can be confirmed objectively. Tinnitus typical signals can be proven and are the basis for an individual therapy. Tinnitus therapies can be evaluated for their effectiveness employing this diagnostic concept. (FIG. 10).
a. EEG Data Collection System and Spectrographic Analysis Evaluating Tool (Cermag)

For the collection and evaluation of the QEEG data a 4-8 channel EEG system is developed. The diagnostic parameters, which provide necessary treatment parameters for the patient, should be integrated in the newly developed system. For this a new software will be necessary, which will developed by AB Neurotech under the direction of NeuroNet and GJB data processing technology GmbH. The software will control the EEG diagnostic system as well as the magnetic field. Since there is no need for a 19 channel system to record the EEG of tinnitus patients, a 4-8 channel system is suitable. The amplification system and the electrodes must be optimized for the measurement in the magnetic field. These new technical possibilities should be easy to handle and to operate. However the development will refer exclusively to the collection and evaluation of the necessary individualized tinnitus treatment parameters.

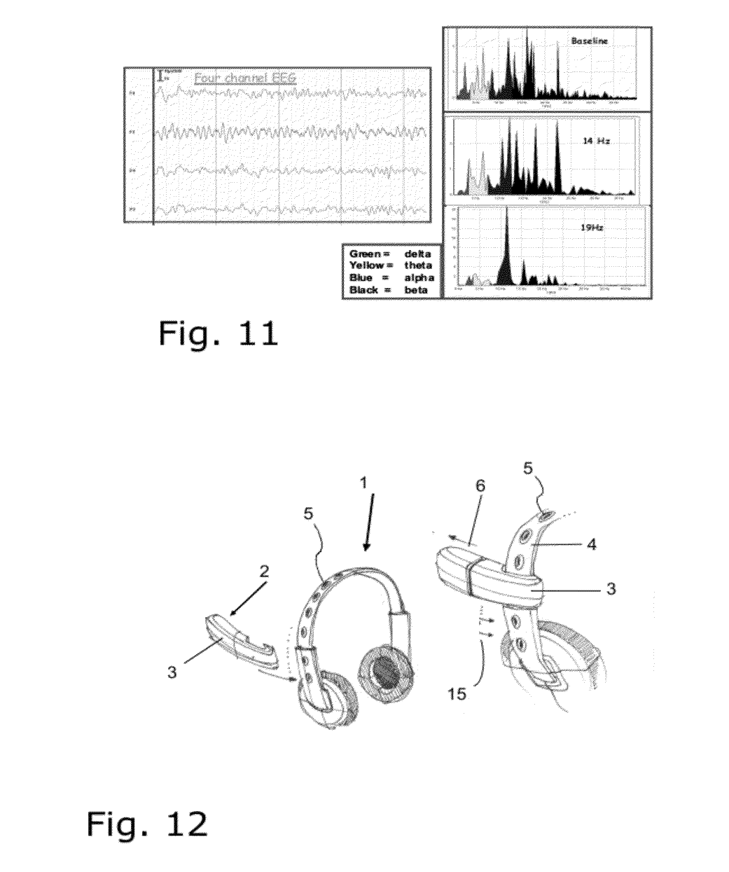

As shown in FIG. 11 both the raw EEG signal and the frequency spectrum should be displayed on the screen.

Baseline: EEG recordings without the application of the magnetic field

14 and 19 Hz: Recording of the brain waves with simultaneous application of magnetic fields with different frequency bands. The values listed indicate the frequency of the pulsed magnetic field. Colors are not necessary but helpful for the user.

In the present literature it could already be shown that the presence of tinnitus induces changes of the electroencephalographic activity. Several research groups were able to demonstrate that tinnitus correlates with altered EEG profiles:

1. Reduction of alpha power

2. Increase of delta power

3. increase of theta power

4. increase of beta power

5. increase of gamma power.

In this context we were able to develop a procedure and a device, with which the above stated abnormalities can be corrected.

In this context a pulsating magnetic field of low field strength, in particular below 20 mT, preferably less than 10 mT, in particular less than 1 mT was employed. A field strength of 5 μT was successfully employed. The frequency of the pulsation ranges between 1 and 100 Hz. This system enables us to select a specific frequency of the magnetic field needed.

### Example

Applying a pulsating magnetic field in with the frequency of e.g. 1 Hz for at least 1 minute; then applying a magnetic field with the frequency of 2 Hz for at least 1 minute etc. while a simultaneous recording of the electroencephalographic activity was performed

Effects of the pulsating magnetic fields on the EEG were demonstrated by power spectrum, brain maps and compressed spectral array (CSA). All technically possible producible pulsating magnetic fields are examined with this technology for their effect on the brain wave pattern. As a positive effect of the pulsating magnetic field the following reactions were identified:

Reduction of the delta power

Reduction of the theta power

Acceleraton of the alpha frequency

Increase of the alpha power

Reduction of the beeta power

Reduction of the gamma power

Negative effects identified being:

Increase of the delta power

Increase of the Theta power

slowing of the alpha frequency

Reduction of the alpha power

Increase of the beta power

Increase of the gammas power.

Of great significance is the vertical and or horizontal application of the pulsating magnetic field.

For both forms of application the effects of the pulsating magnetic fields are represented by means of a power spectrum, brain map and a CSA.

Decision making aids for magnetic field therapy

Increase of the alpha power

Acceleration of the alpha frequency

Reduction of the beta power

Reduction of the gamma power

Reduction of the delta power

Reduction of the theta power

alpha/beta ratio (therapy)>alpha/beta ratio (baseline)

alpha/gamma ratio (therapy)>alpha/gamma ratio (baseline)

alpha theta ratio (therapy)>alpha/theta ratio (baseline) alpha/delta ratio (therapy)>

alpha/delta ratio (baseline)

Delta: 2-4 Hz

Theta: 4-7 Hz

Alpha: 8-13 Hz

Beta; 14-21 Hz

low gamma (30-42 Hz)

40 Hz (38-42 Hz)

mid gamma (43-63 Hz)

high gamma (64-100 Hz)

It is intended to develop an objective diagnostic test tool for tinnitus that is economical, simple and fast. If a stimulus, for example noise or a tone, is registered by the brain, brain waves with a frequency 40 Hz can be detected. This brain wave is called gamma activity. This activity is only present during a stimulus; thus during a tone or noise. If someone constantly notices tinnitus, gamma activity should be constantly present in the auditory cortex. The louder the tinnitus, the higher audio range activities should be present. Gamma activity is determined by use of the EEG equipment which seize objectively Tinnitus in its presence and its intensity. Second effort is to observe brain activity while employing fMRI technology. These pictures were employed to identify tinnitus and/or tinnitus intensity. If one is able to measure objective tinnitus one can use the same procedure and the same technology, in order to treat the disturbances caused by tinnitus.

With the help of the quantitative EEG subjective feelings of a patient can be confirmed objectively Tinnitus relevant signals can be demonstrated, and are the base for an individual therapy. The success of a tinnitus therapy can be evaluated with this diagnostic tool

b. Therapy Equipment Certis

Significant advances in the development (FIG. 5):

Hardware:

Construction of Base
minimize Size
formal-aesthetic Design
durable construction of the device
simple handling
LCD display
touch screen
battery operation
simple change of battery
simple cleaning
fulfillment of the medicine-technical regulations
timer function
international labeling
no standard socket
on back: international labels for necessary product data
equipment should not be opened by unauthorized persons with standard tool.

Functions of the Display
display program
display power on/off
timer
battery control
menu guidance

Construction of Head Device
optimized electrical field: Frequency, amplitude, flow density
optimized positioning of a a applicator at the mastoid for a period of 20 up to 60 minutes daily for one period of up to 24 months
individual adaptability to different head forms and—sizes
pleasant in wearing
simple handling—even for older and technically not experienced humans
during sleep—may not be damaged, if the patient thereby falls asleep
simple to clean
formal-aesthetic Design
control functions at the head device
international symbol labelling
connection to the base with a not standardized socket
must fulfill all requirements for medical permissions (e.g. CE, FDA, etc.)
endurable construction—no parts which can be damaged easily
maintenance-free

Software

Playing of Tinnitus Music-Therapy Audio-Files from a Memory Card (Max. 8 GB)
Graphic display with touchscreen:
   program display
   time display
   power on off display
   battery control display
Best high audio quality possible without loss of data (no MP 3 format)
Headphones and equipment should be adjusted concerning the impedance headphone, in order to achieve optimal acoustic transmission characteristics
Copy protection: Content of the equipment not downloadable, Watermarking-able (Frauenhofer system), but no DRM 10-10-able copy protection
Own data format! Data delete themselves automatically upon expiration of the subscription
Compliance control: Frequency and time of listening can be determined at a later point in time, built-in counter permits to track down day and hours when music was listened to. Identification of the patient's parameters with which individual adjustments of the equipment parameters can be made.
Firmware and music data can be transmitted via PC
Fulfillment of the medical-technical regulations
Equipment cannot be opened by unauthorized persons with standard tools, seal on data medium—damage can be detected.
On the backside: international label for necessary product data

Hardware

### Circuit Board and Housing

The core of the equipment is the graphic display with a touchscreen. The operation is very similar to an “IPod”. In addition it comes still that the equipment except by disassembly of the integrated memory module, on which the data are or by splitting the headphone cables (! analogue copy!) is digitally copy-protected, since no usual interfaces for PC's are not used and the data file format of the music raw data a usual file format are.

In the following section the technical data of the hardware are described briefly:
Plastic housing (hand device)
Graphic display backlight color white with touchscreen; construction units SMD, 3.3V-technics
2 alkaline batteries type AA, potential transformer with high efficiency
Power down switch
Reverse voltage protection
Standard socket 3.5 mm
Data storage:
   MicroSD card, can be plugged in; File system FAT16/32 (PC compatible); Option: 2. MicroSD card for a log file (application times, duration, etc.)
Controller:
   Microchip signal processor (dsPIC33F, to 40MIPS)
Audio exit:
   44.1 or 48 kHz sampling rate, 16 bits per channel, stereo; volume and bass/pitch adjustment; headphone exit 2×35 mW
Clock/calendar:
   RTC with 32 kHz-clock, lithium backup button cell (CR1220)
Equipment assembled, programed and tested, without micro SD maps and batteries.

The system software (hardware driver) is written in C (Microchip compilers), with additional libraries.

The actual battery life span can only be determined after practice run; assumed life span >50 h. If needed compatible accumulators can be used, since the potential transformer starts functioning with 1V.

The SD-cards-LIBRARY is already SDHC compatible; approximately 1200 minutes are possible with 48 kHz/stereo e.g. with a 16 GB microSD map (there are also bigger cards!).

A head device is developed, which is aligned to the specific requirements of application. Thereby following points need to be considered:

Improved Fitting:

The head device will be able to adapt itself to the different head forms and—sizes. Due to the special construction, the applicator is placed more precisely at the desired site e.gg the mastoid. However it is possible that research will show that other sites on the head need to be stimulated. The head device should be very comfortable when worn. It will be tested whether a covering of the ear is reasonable.

Stimulation Sites:

Up to now we believe that the optimal stimulation is performed over the mastoid. If other sites for stimulation are identified, they are to be integrated into the design of the form of the head device.

Possible sites were an effect can be obtained:

A. Inner ear/stria vascularis/ganglion spiral

B. Hearing nerve

C. auditory pathway

D. Brain/Lateral Lobe

Parameter for the determination of the optimal stimulation site:

A differentiation is made between so called psychophysical hearing examinations as threshold examinations and over threshold tonal hearing examinations, as also as language audiometry. With these methods it will be tried to determine the approximate auditory threshold and some typical hearing disorders.

In the presence of central hearing reduction and ear noises objective methods of the hearing examination are used.

Ear

tympanometry—impedance audiometry—electrocochleography

Brain stem

acoustically evoked brain stem potentials ABEP

Cortex

Brain Electrical Activity Mapping (BEAM) with acoustic stimulation.

Virtual Reality Brain Electrical Activity Mapping (VR BEAM)

Brain Electric Tomography

With the help of this gradated technology it is possible to locate the hearing disorders topographically, and thereby determining the kind of hearing disorder. This allows designing an individual therapy. Beside acoustically evoked potentials further suitable measuring procedures are employed: HRV, skin resistance, skin conductance

FIGS. 12-17 show further, possible designs of the head device.

FIG. 12 shows: A head device 1, including a head phone having a holder viz. head phone frame 4 which is equipped with a pushbutton arrangement and a therapy element viz. applicator 2 which is arranged on an arm 3. The entire headphone holder viz. frame 4 is equipped with pushbuttons 5 along an outside. The therapy element 2 is fastened on the ideal height to the frame 4 and can additionally be pulled out along the longitudinal direction, see extendable part 6.

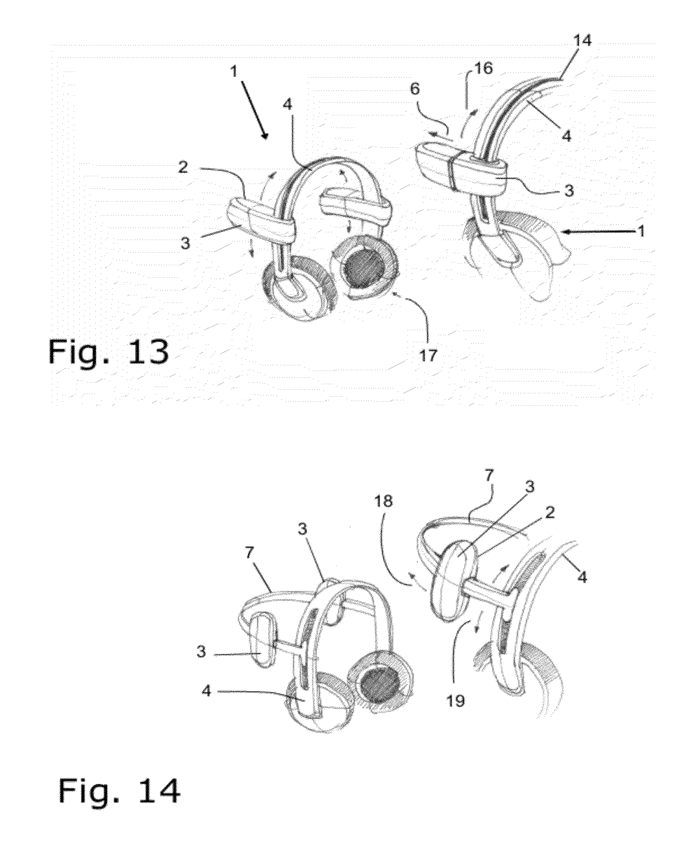

FIG. 13 shows: At the holder 4 of the head device 1 a rail system viz. guide with several rest positions is implemented, at which the therapy element 2 can be adjusted vertically in its height. In addition the therapy element 2 can be pulled horizontally apart and extended thus, in order to achieve the perfect site at the head.

FIG. 14 shows: A transverse handle 7 with two therapy elements 2 is attached at a headphone frame 4 with rail system or guide system 8. Thus the handle 7 can to be shifted along the rail 8 vertically and the therapy elements 2 themselves may be shifted along the transverse handle horizontal to the suitable position. Both sides of the headphone frame 4 and transverse handles 7 can be size-adjusted.

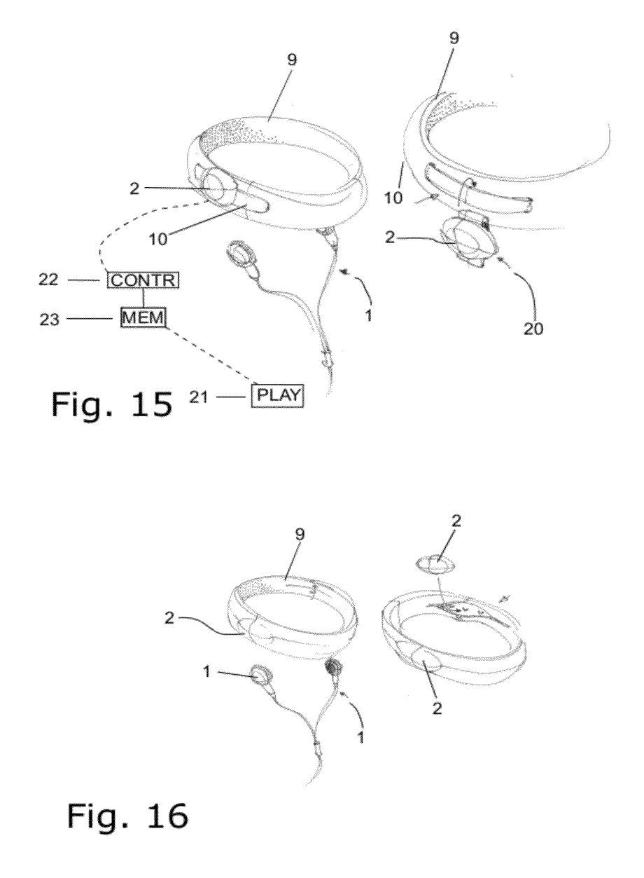

FIG. 15 shows: Both therapy elements 2 are fastened at the left and at the right to a flexible head band 9. The elements 2 can be fastened to a loop 10 of the head band 9 and shifted along the outside lying loop 10 to several positions.

FIG. 16 shows: Both therapy elements 2 are on the right of and left within a flexible head band 9. The head band can be opened on the inside, in order to hang in the elements 2 at different positions.

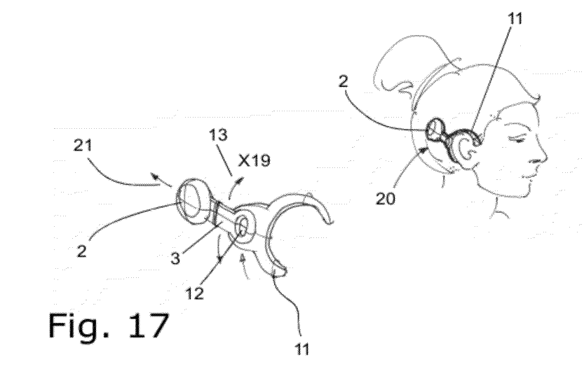

FIG. 17 shows: The therapy device is attached to an ear clip 11, which is placed outside on the external ear and can be clamped behind the ear as shown. The element 2 comprises a housing of a coil is fastened to a movable arm 3, shown left in the figure, in order to make a vertical positioning possible. In addition the arm 3 can be extended in order to permit a horizontal positioning.

Functionality of the Stimulation Device:

Operation: The new equipment will be operated via three “push button switches”. The functions are visible on an LCD.

Menu: Several programs can be selected. The control menu will guide the use.

Programming: Via interface the parameters/programs can be altered. It is to be considered whether it is a direct interface or whether one a change can be introduced in a later generation over a PC. The equipment will be programmed in a way that a person can perform a treatment per day. A temporarily more intensive use by several persons is thus prevented. For physicians a special programmed device on license basis will be offered.

Timer & Compliance monitor: Time registration and control system for the memory and simple application with the goal minimizing the time to the necessary minimum. Identification of patient parameters on the basis of an individual adaption of the parameters of the device is possible.

Labelling: The entire equipment is provided with an international symbol inscription.

Appearance:

Design: A functional-aesthetic design improving the technical competence and the reliability of the device.

Control of functions: At any time the user should be enabled to convince himself of the optimal functioning of the basis and head device.

Packing: It will come with a suitable transportation packing and an appropriate packing for the device (e.g. casket).

Application:

Patient selection: evaluation of inclusion and exclusion criteria with the goal of receiving a homogeneous patient pool.

Manual & Therapy support: Optimal support of the patient before/during/after the treatment.

Objective result: Based on the present knowledge it remains our goal to integrate a system for a direct and/or indirect evaluation of the therapy e.g. via measurement of objective parameters

The following appears to be important:

Q-EEG is the basis diagnostic tool

The testing of the individual effect of pulsed magnetic fields in the frequency range 1 Hz up to 100 Hz, preferably 1 to 19 Hz on the tinnitus-related signals in the QEEG.

The testing of the individual effect of pulsed magnetic fields in the frequency range 1 Hz up to 100 Hz, preferably 1 to 19 Hz on all Q-EEG recordings.

The testing of the individual effect of pulsed magnetic fields in the frequency range 1 Hz up to 100 Hz, preferably 1 to 19 Hz illustrated in form of brain maps.

The testing of the individual effect of pulsed magnetic fields in the frequency range 1 Hz up to 100 Hz, preferably 1 to 19 Hz illustrated in form of power spectrum.

The application of pulsed magnetic fields on the basis of the individual QEEG results.

The use of alpha state promoting music during simultaneous application of pulsed magnetic field.

The use of alpha state promoting music during simultaneous application of pulsed magnetic fields based on the individual test results.

The application of tonal signals, which according to the result of the examination pulsating magnetic fields cause a frequency consequence action with same frequency as the magnetic field

The application of tonal signals, which according to the result of the examination pulsating magnetic fields cause a frequency consequence action with same frequency as the magnetic field with simultaneous use of alpha conditions of promoting music

The application of pulsed magnetic fields on the basis of the individual results of the quantitative EEG investigation combined with simultaneous visual stimulation with defined frequency bands

The use of alpha state promoting music during simultaneous application of pulsed magnetic fields combined with simultaneous visual stimulation with defined frequency bands.

The use of alpha state promoting music during simultaneous application of pulsed magnetic fields according to the individual result of measurement combined with simultaneous visual stimulation with defined frequency bands

The application of tonal signals, which according to the result of the examination pulsating magnetic fields cause a frequency consequence action with same frequency as the magnetic field combined with simultaneous visual stimulation with defined frequency bands

The application of tonal signals, which according to the result of the examination pulsating magnetic fields cause a frequency consequence action with same frequency as the magnetic field with simultaneous use of alpha conditions of promoting music combined with simultaneous visual stimulation with defined frequency bands

The application of visual stimulation on the basis of the QEEG investigation

The application of visual stimulation based on QEEG results with simultaneous application of pulsed magnetic fields.

The application of signals in a technical composition in shape, descriptive above that Magnetfeldapplikator and those auditi yen signals transferring loudspeakers in a common housing are installed, that the technical signal carrier is installed in a head frame 4 that at the head frame 4 several signal carriers to be installed can that at the head frame 4 several signal carriers are adjustably installed.

### Patent Idea 2

Music and frequency following response in the electrical activity of the brain frequency.

The idea is that music of the compose Mozart is offered in stereo. For example a tone with 400 Hz should be recorded on the right channel, and a tone with 410 Hz should be recorded on the left channel. Same approach should be performed with other frequencies, (examples: 400-401, 400-402, 400-411, 400-416 Hz etc.,) in order to be able to induce a Frequency Following Response according to the determined magnetic field frequency.

Thus the magnetic field therapy is combined with a auditory stimulation, which should strengthen mutual effects. For this combination protection is requested.

Goal of the Music Therapy:

Introduction: The researcher Robert Monroe discovered that the Frequency Following Response is not limited to the region of the brain range that is responsible for hearing or only in the left or only the right hemisphere. The phenomenon could be detected throughout the brain. The brain waves were identical regarding frequency, amplitude, phase and coherence. Monroe discovered a technology for the production of hemisphere synchronization. After stimulation within the theta frequency range, his test persons experienced: hypnagogic state, creative thoughts, integrative experiences and spontaneous memory pictures. Stimulation within the beta range led to improved attention and concentration. Monroe obtained a patent and called this procedure HemiSync (registered trademark). Nowadays HemiSync is used world-wide for therapeutic purposes as well as using it at home.

This concept, seemingly discovered by scientists of the modern time, has already been used by medicine men and shamans for many years. The rhythmic sound of the drum is a fundamental instrument for the release and maintenance of the shamanic consciousness state. Research data revealed that the regular, monotonous drum rhythm has a dramatic impact on the electroencephalographic activity. Observers of shamanistic rituals stated that during the period of initiation of the rites Theta waves dominated.

Goals:

Induction of 12 Hz alpha activity is induced by offering two tones with a difference of 12 Hz.

(1012 Hz 1000 Hz, f1-f2) the brain notices 12 Hz.

Adjusted Frequency Range:

a) within the tinnitus range, adapted to the individual tone audiogram, about 20 dB, thus clearly audibly

b) not within the Tinnitus frequency range in order to achieve an optimal hearing and a generation of 12 Hz

Induction of a frequency range detected via Q-EEG by means of magnetic field detected frequency range shows in the QEEG that for the patient a slow frequency band is necessary e.g., possibly 3 Hz, then the computation would be as follows: The patient should be offered two tones, which generate 3 Hz, also here again in either the audiometrically measured tinnitus region or within a range with optimal hearing perception. (f1-f3 results in 3 Hz, 1003 cycles tone and 1000 cycles per second tone)

Tones from the low frequency range, in order to along-use the feeling perception, or tones from the middle or high frequency range. A bass tone stimulation with low frequencies is lasting and can lead to therapeutic effects. One of the decision criteria should be the audiogram, but if the patient concerned does not have serious hearing loss, what to do then? May then tones from to the tactile range be used or should the tones be made dependent from what is pleasant to the patient and/or resp. that the music offered, to which tones are added, does not disturb too much? Remedy may be obtained by an oto-acoustical measurement.

Signal Forms:

It is claimed that the therapy equipment and the diagnose unit can use all possible stimulation signal samples for the optimization of the QEEG of results, in particular with electromagnetic or electrical stimulation. There are no restrictions of the desired and possible signal form, with which stimulation can be performed.

Other Stimulation Procedures:

The protection sought for the Certis& Cermag System includes the evaluation and optimization by QEEG of all other known different stimulation procedures like e.g. also audiovisual or visual, thermal stimulation, with laser & rocking systems for stimulation optimization, all this is possible with Certis& Cermag.

1. A device for a tinnitus therapy comprising EEG equipment and a head device having at least one applicator adapted to produce a low-frequency electromagnetic field within a frequency range of 1-100 Hz with a field strength below 20 mT.

2. A device according to claim 1, wherein the head device further comprises a holder adapted to hold the head device on a head of a patient, and wherein the applicator is adjustably connected to the holder.

3. A device according to claim 1, wherein the head device further comprises a device for emitting at least one of sound and music.

4. A device according to claim 1, wherein the field strength of the electromagnetic field of the applicator is below 1 mT.

5. A device according to claim 1, wherein the EEG equipment differentiates a subdivision of recorded EEG signals into frequency bands.

6. A device according to claim 2 wherein the holder comprises at least one of a headphone frame, a head band, an ear clip and a head cap.

7. A device according to claim 1, further comprising a playback device for reproducing sound.

8. A device according to claim 1, wherein the operating frequency of the applicator is adjustable.

9. A device according to claim 1, further comprising a control unit having a memory chip for at least one of the operating frequency of the applicator and the field strength of the applicator.

10. A device according to claim 1, wherein the EEG equipment is qualitative EEG equipment.

11. A device according to claim 1, wherein the field strength of the electromagnetic field of the application is below 0.2 mT.

12. A device according to claim 1, wherein the field strength of the electromagnetic field of the application is below 0.1 mT.

13. A device according to claim 1, wherein the frequency is adjustable by a patient.

14. A device according to claim 3, wherein the device for emitting at least one of sound and music is a headphone.

15. A device according to claim 5, wherein the EEG equipment differentiates a subdivision of the recorded EEG signals into delta, theta, alpha, beta and gamma frequency bands.

16. A device according to claim 7, wherein the sound is music.
